SEQUENCE LISTING (11 sequences)

SEQ ID NO: 1 - DNA, 990 bp, Artificial Sequence
  feline IL-12 p40
atgcatcctcagcagttggtcatcgcctggttttccctggttttgctggcacctcccctcatggccatatgggaactggagaaaaacgtttatgttgtagagttggactggcaccctgatgcccccggagaaatggtggtccttacctgcaatactcctgaagaagatgacatcacctggacctctgaccagagcagtgaagtcctaggctctggtaaaactctgaccatccaagtcaaagaatttgcagatgctggccagtatacctgtcataaaggaggcgaggttctgagccattcgttcctcctgatacacaaaaaggaagatggaatttggtccactgatatcttaagggaacagaaagaatccaaaaataagatctttctaaaatgtgaggcaaagaattattctggacgtttcacctgctggtggctgacggcaatcagtaccgatttgaaattcactgtcaaaagcagcagaggctcctctgacccccaaggggtgacttgtggagcagcgacactctcagcagagaaggtcagagtggacaacagggattataagaagtacacagtggagtgtcaggagggcagtgcctgcccggctgccgaggagagcctacccattgaagtcgtggtggacgctattcacaagctcaagtacgaaaactacaccagcagcttcttcatcagggacatcatcaaaccggacccacccaagaacctgcaactgaagccattaaaaaattctcggcatgtggaagtgagctgggaataccctgacacctggagcaccccacattcctacttctccttaacatttggcgtacaggtccagggcaagaacaacagagaaaagaaagacagactctccgtggacaagacctcagccaaggtcgtgtgccacaaggatgccaagatccgcgtgcaagccagggaccgctactatagctcatcctggagcaactgggcatccgtgtcctgcagttag

SEQ ID NO: 2 - DNA, 669 bp, Artificial Sequence
  feline IL-12 p35
atgtgcccgccgcgtggcctcctccttgtaaccatcctggtcctgttaaaccacctggaccacctcagtttggccaggaacctccccacacccacaccaagcccaggaatgttccagtgcctcaaccactcccaaaccctgctgcgagccatcagcaacacgcttcagaaggccagacaaactctagaattttacccctgcacttccgaagagattgatcatgaagatatcacaaaagataaaaccagcacagtggaggcctgcttaccactggaattagccatgaatgagagttgcctggcttccagagagatctctctgataactaatgggagttgcctggtgtccagaaagacctcttttatgacgaccctgtgccttagcagtatctatgaggacttgaagatgtaccaggtggagttcaaggccatgaatgcaaagctgttaatggatcctaaaaggcagatctttctggatcaaaacatgctgacagctattgatgagctgatgcaggccctgaatttcaacagtgtgactgtgccacagaactcctcccttgaagaaccggatttttataaaactaaaatcaagctctgcatacttcttcatgctttcagaatccgtgcagtgaccatcaatagaatgatgagctatctgaatgcttcctag

SEQ ID NO: 3 - DNA, 74 bp, Artificial Sequence
  5′-Primer
gagagttctcagagctcctaactgcaggacacggatggagagttctcagagctcatcctgggggtggaacctaa

SEQ ID NO: 4 - DNA, 37 bp, Artificial Sequence
  5′-Primer
gtagcggataaggtaccatgcatcctcagcagttggt

SEQ ID NO: 5 - DNA, 37 bp, Artificial Sequence
  5′-Primer
gagagttctcagagctcatcctgggggtggaacctaa

SEQ ID NO: 6 - DNA, 76 bp, Artificial Sequence
  Primer fIL12-p35 (eco-)r
gagagttctcagagctcctaggaagcattcagatagctcatcattctattgatggtcactgcacggattctgaaag

SEQ ID NO: 7 - DNA, 37 bp, Artificial Sequence
  Primer fIL-12p35-1
gtagcggataaggtaccatgtgcccgccgcgtggcct

SEQ ID NO: 8 - DNA, 71 bp, Artificial Sequence
  Primer f12p35-1-lang
tgctgacagctattgatgagctgttacaggccctgaatgtcaacagtgtgactgtgccacagaactcctcc

SEQ ID NO: 9 - DNA, 76 bp, Artificial Sequence; identical to SEQ ID NO: 6
  Primer fIL12-p35(eco-)-r

SEQ ID NO: 10 - DNA, 4522 bp, Artificial Sequence
  pMol-fil12p40
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggtctccccctggatccgctagcttaaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatggtaccatgcatcctcagcagttggtcatcgcctggttttccctggttttgctggcacctcccctcatggccatatgggaactggagaaaaacgtttatgttgtagagttggactggcaccctgatgcccccggagaaatggtggtccttacctgcaatactcctgaagaagatgacatcacctggacctctgaccagagcagtgaagtcctaggctctggtaaaactctgaccatccaagtcaaagaatttgcagatgctggccagtatacctgtcataaaggaggcgaggttctgagccattcgttcctcctgatacacaaaaaggaagatggaatttggtccactgatatcttaagggaacagaaagaatccaaaaataagatctttctaaaatgtgaggcaaagaattattctggacgtttcacctgctggtggctgacggcaatcagtaccgatttgaaattcactgtcaaaagcagcagaggctcctctgacccccaaggggtgacttgtggagcagcgacactctcagcagagaaggtcagagtggacaacagggattataagaagtacacagtggagtgtcaggagggcagtgcctgcccggctgccgaggagagcctacccattgaagtcgtggtggacgctattcacaagctcaagtacgaaaactacaccagcagcttcttcatcagggacatcatcaaaccggacccacccaagaacctgcaactgaagccattaaaaaattctcggcatgtggaagtgagctgggaataccctgacacctggagcaccccacattcctacttctccttaacatttggcgtacaggtccagggcaagaacaacagagaaaagaaagacagactctccgtggacaagacctcagccaaggtcgtgtgccacaaggatgccaagatccgcgtgcaagccagggaccgctactatagctcatcctggagcaactgggcatccgtgtcctgcagttaggagctcataatcagccataccacatttgtagaggttttacttgctttaaaaaacctcccacacctccccctgaacctgaaacataaaatgaatgcaattcttgttgttaacttgtttattgcagcttataatggttacaaataaagcaatagcatcacaaatttcacaaataaagcatttttttcactgcattctagttgtggtttgtccaaactcatcaatgtatcttaacgcgaattcagggggagacccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 11 - DNA, 20 bp, Artificial Sequence
  5′-phosphorylated nucleotide
aggggtccagttttctggac

(19) United States
(12) Patent Application Publication
(10) Pub. No.: US 2003/0157059 A1
(43) Pub. Date: Aug. 21, 2003

(54) Feline interleukin-12 as immunostimulant

(76) Inventors: Hans Lutz, Rudlingen (CH); Christian Leutenegger, Uster (CH); Nils Pedersen, Winters, CA (US); Matthias Schroff, Berlin (DE); Burghardt Wittig, Berlin (DE)

Correspondence Address: NILS H. LJUNGMAN, ESQUIRE, NILS H. LJUNGMAN & ASSOCIATES, P.O. BOX 130, GREENSBURG, PA, 15601-0130, US

(21) Appl. No.: 10/041,672

(22) Filed: Jan. 8, 2002

Related U.S. Application Data
(63) Continuation-in-part of application No. PCT/DE00/02263, filed on Jul. 8, 2000.

(30) Foreign Application Priority Data
Jul. 8, 1999 (CH) 1259/99

(51) Int. Cl. A61K 48/00; A61K 38/20

(52) U.S. Cl. 424/85.2; 514/44

(57) ABSTRACT

The use of feline interleukin-12 (IL-12) is described as an adjuvant (or immunostimulant) and in the vaccination and therapy of infectious diseases in Felidae. In addition, a procedure is disclosed which makes it possible to express the two subunits of IL-12 in the requisite mass ratio.

### CONTINUING APPLICATION DATA

This Application is a Continuation-in-Part of International Patent Application No. PCT/DE00/02263, filed on Jul. 8, 2000, which claims priority from Swiss Patent Application No. 1259/99, filed on Jul. 8, 1999. International Patent Application No. PCT/DE00/02263 was pending as of the filing of this application. The United States was an elected state in International Patent Application No. PCT/DE00/02263.

### BACKGROUND OF THE INVENTION

1. Field of the Invention

The invention describes the feline cytokine interleukin-12 (IL-12) and its use as immunostimulant in the Felidae.

2. Background Information

Interleukin 12 (IL-12) is one of the group of substances known as cytokines, a group of proteins which transmits signals between different cells that participate in the coordination and execution of the immune response. IL-12 was published under the name of “Natural Killer Cell Stimulatory Factor” (Trinchieri et al. in EP 0 441 900, U.S. Pat. No. 5,571,515). Interleukin 12 is a heterodimeric protein, consisting of the subunits p35 and p40. IL-12 is one of the class of cytokines involved in the first phases of the immune response and closely related to the systems of natural immunity (macrophages, complement). It has a decisive influence on the type of adaptive immune response which develops, as it is one of the so-called “Type 1” cytokines, which support the development of a cytotoxic response based on T-cells. The properties of IL-12 include the stimulation of the secretion of interferon-γ by CD4-positive helper T-cell population. On the basis of these properties, IL-12 may be suitable as an immune adjuvant or as an immunostimulant for the cure of diseases which are already present.

Natural protection against infectious diseases is based on the recognition by the immune system of structures in pathogens which have already been successfully combated. Two main activities may be distinguished here. On the one hand, there is the activity of the humoral immune system. This is based not only on the synthesis of antibodies by plasma cells, formed from B-lymphocytes, but also on humoral components of non-adaptive natural immunity, such as the complement system. Antibodies are soluble protein molecules which are capable of binding specifically to antigens, which may be either soluble or on the surface of cells, bacteria or viruses and accessible to antibodies. As a result of complex formation with antibodies, the pathogens or toxins are either inactivated or put in a form which is recognisable to components of the natural immune system, which then remove it. The second branch of the immune system is the cellular immune system, which is based predominantly on the activity of T-lymphocytes, but also on “natural killer cells” and the antigen-presenting cells of the natural immune system. T-lymphocytes are capable of recognising body cells infected with viruses as “foreign”, if the infected cells present suitable structures which are recognisable by the T-cells. Depending on their specific function, T-cells either amplify and modify this signal for recognising foreign structures (T helper cells, so-called CD4⁺ cells) or directly induce the lysis of the cell which has been recognised as foreign or infected (cytotoxic T-cells, so-called CD8⁺ cells). Correct cooperation between the humoral and cellular immune systems is of decisive importance for the function of the immune response. In the last ten years it has become clear that the cellular arm of the immune system is induced by activation of the so-called type 1 helper cells and the humoral arm by activation of the so-called type 2 helper cells (Mosmann et al., 1986). In keeping with this, the cellular arm is also known as the “TH1 pathway” and the humoral arm as the “TH2 pathway” of the immune system. Bacteria are combated mostly by the TH2 pathway, in which antigenic binding sites on the surface of the bacterium are covered with antibodies. Bacteria coated in this way can then be eliminated by phagocytic cells. The TH2 pathway is-also important for the neutralisation of bacterial toxins and for combating certain parasites found in the extracellular space in the body of the patient. On the other hand, pathogens which live intracellularly, as is the case for certain bacteria and all viruses, are combated mostly by the TH1 pathway of the immune system, i.e. with cytotoxic T-cells. Some pathogens stimulate only one pathway of the immune system, so that certain diseases which result in the stimulation of only the TH2 pathway cannot usually be controlled by the immune system, or at best not efficiently. In such cases, stimulation of the TH2 immune response by vaccination is also ineffective. Induction of the TH1 immune response after vaccination is only possible if the vaccine antigen can replicate itself. Vaccines which cannot replicate in the animal can only induce a humoral but not a cellular immune response.

Several years ago it was demonstrated that one of the factors affecting the induction of a TH1 immune response is the synthesis of IL-12. After this had been recognised in the mouse, a number of research groups began to look for IL-12 in the cat, as cats suffer from several infectious diseases which can only be overcome with a functioning TH1 immune system. Examples include infections with the feline immune deficiency virus (FIV), feline leukaemia virus (FeLV) and feline coronavirus (FcoV).

A sequence of the p35 subunit of feline IL-12 was published in 1994 (Bush K, et al. 1994). The complete sequence was determined in 1996 and published in 1997 (Fehr et al., 1997; Schijns et al., 1997). After these publications the next step was to examine the function of IL-12 in vivo, in particular whether IL-12 has the same functions in directing the developing immune response. In spite of considerable effort on the part of several research groups devoted to this question, it has not yet been possible to demonstrate the functional activity of the published sequence or to demonstrate its function as an immune stimulant. It is known that in other animals the two subunits of IL-12 must be formed simultaneously in the correct ratio in the same cell for functional IL-12 to be formed (Picofti et al. 1997). This was not successfully achieved in previous experiments with feline IL-12. An even more fundamental hurdle lay in the difficulties, which have never been explained, in cloning a functional recombinant sequence of feline p35 in E.coli. Despite the facts that the sequence of recombinants representing partial sections of the whole sequence has been known for years and that feline IL-12 is very similar to the human and bovine proteins, it has not yet been possible to isolate a clone of the complete p35 sequence.

### OBJECT OF THE INVENTION

Starting from this current state of scientific knowledge, it is the purpose of the present invention to make available a functional feline IL-12 and/or the necessary sequence in the form of nucleic acids, and in this way to induce a TH1 immune response in the target cells of Felidae, via synthesis of interferon-γ or other biologically active molecules.

### SUMMARY OF THE INVENTION

The problem has been solved in the invention as follows. The methods of recombinant gene expression were used to express the two polypeptide chains of subunits p35 and p40 of feline interleukin 12 in eukaryotic or prokaryotic cells. The proteins formed are then extracted so that they can be used in equimolar concentrations in the presence of an antigen which is suitable for immunisation. It is not important whether the antigen is administered with the IL-12 by co-injection or other forms of external administration, or whether the antigen is already present in the (non-human) animal (such as a cat) which is to be treated, as the result of an already existing disease or allergy, and contributes locally to the development of the required response.

Alternatively, vaccination using the adjuvant or immunostimulatory activity of the present invention can also be achieved by administering into the cells of the cat one or more DNA constructs consisting of genes which code for the p35 and p40 subunits of feline IL12 and which are controlled by the promoter, terminator or polyadenylate sequences that operate in the cat. These then trigger the synthesis of functional IL-12 and thus the desired sequence of immunostimulatory signals, particularly γ-interferon. Further aspects of the invention are immunostimulation for the treatment of certain diseases or an adjuvant for co-injection with antigen.

Within the framework of the invention, IL-12 can be used as an adjuvant or immunostimulant to treat diseases in which a TH1 response is helpful. This has already been postulated for some animal species and for man (Gately and Mulqueen, 1996), but has not yet been successfully demonstrated. Examples of diseases in which feline IL-12 in the present invention could be used as adjuvant include infection with feline coronavirus, which leads to the feared and widespread feline infectious peritonitis (FIP). In this disease, for reasons which are not yet clear, there is a massive predominance of the TH2 response, which leads to vasculitis, peritonitis and death. That this occurs had long been presumed, but was confirmed by measurements of cytokine activity in cats suffering from FIP. All cytokines specific for TH2 were detected in excess, but IL-12 and γ-interferon were found only in negligible amounts, if at all. Other examples are infections with FIV or with feline leukaemia virus (FeLV). These two retrovirus infections are characterised by the intracellular presence of the virus, and thus elude a humoral immune response. Stimulation of the TH1 response by treatment with IL-12 makes it possible to remove, or considerably to reduce, the amount of virus in infected cats. These are only a few examples; the list is not exhaustive.

In general, the present invention includes polypeptides with at least 95% sequence homology with the polypeptides coded by the nucleotide sequence fIL12p40 (p40 subunit of feline IL-12: SEQ ID NO 1) and fIL12p35 (IL-12SEQ ID NO 2), as immunostimulants, particularly for the prevention and treatment of disease in carnivores, specifically the domestic cat.

Nucleic acid constructs which contain sequences with at least 95% sequence homology to the sequences of fIL12p40 (p40 subunit of feline IL-12: SEQ ID NO 1) and fIL12p35 (p35 subunit of the feline: SEQ ID NO 2) and in which the sequences are under the control of promoter and terminator sequences which operate in higher animals such as carnivores, particularly Felidae and specifically the domestic cat, are, according to the invention, suitable immunostimulants for immunisation against infectious diseases and for the treatment of infectious diseases and tumours in carnivores, in particular Felidae and specifically the domestic cat. These nucleic acid constructs are chiefly those in which the construct consists of linear double-stranded DNA, which is covalently bonded at both ends and which contains only one promoter and the coding sequence in each strand.

A polypeptide in accordance with this invention is suitable for the treatment of tumours and of autoimmune diseases and of diseases in which there is a TH1 deficiency, as particularly in established infections with FIV, FeLV and coronavirus.

A nucleic acid construct in accordance with the invention is suitable as adjuvant for prophylactic immunisation against viral infection of carnivores, particularly Felidae and specifically the domestic cat, and specifically for immunisation against FIV infection and/or immunisation against FeLV infection. In addition, a nucleic acid construct in accordance with the invention is suitable for the treatment of diseases in which there is a TH1 deficiency, particularly when infection with FIV, FeLV or FcoV is present.

Further advantageous procedure are contained in the other sub-claims. The invention will now be explained more closely on the basis of some embodiments which have been performed and in the figures below. These embodiments are intended only to improve the understanding of the invention, without restricting it in any way.

The above-discussed embodiments of the present invention will be described further hereinbelow. When the word “invention” is used in this specification, the word “invention” includes “inventions”, that is the plural of “invention”. By stating “invention”, the Applicant does not in any way admit that the present application does not include more than one patentably and non-obviously distinct invention, and maintains that this application may include more than one patentably and non-obviously distinct invention. The Applicant hereby asserts that the disclosure of this application may include more than one invention, and, in the event that there is more than one invention, that these inventions may be patentable and non-obvious one with respect to the other.

### BRIEF DESCRIPTION OF THE DRAWINGS

The invention is explained in greater detail below with reference to “the accompanying drawings.”

FIG. 1 illustrates the cloning strategy used for the p35 construct. It shows how the feline p35 gene is stably complemented by sequences of human IL-12 p35.

FIG. 2 is a comparison between the sequences of human and feline p35. Legend:

Line 1: Human sequence pG-hIL12p;

Line 2: Feline sequence according to Fehr et al.; fil12p35a;

Line 3: Recombinant sequence pMOL-fIL12 p35;

Line 4: Left primer of the 3′fragment;

Line 5: Right primer of the 3′ fragment

FIG. 3 shows an IRES construct, in which the construct is preceded by CMV and T7 promoter and important restriction sites are shown.

FIG. 4 shows the expression of γ-interferon (IFN) in lymphocytes after incubation with supernatants from 3201 cells which had previously been transfected with different polynucleotides. p35E: p35 introduced by electroporation; IL12p35p40E: complete IL-12 introduced by electroporation; IL12p40E: p40 introduced by electroporation; GFP BT: ballistically transfected GFP gene; IL12p35p40 BT: ballistically transfected complete IL-12; GFP-E: GFP gene introduced by electroporation.

FIG. 5 shows the RNA virus load at week 5 in animals in the 3 groups.

### DESCRIPTION OF THE PREFERRED EMBODIMENT

The essence of the invention is the preparation of both subunits of IL-12 in a form which makes it possible for them to be expressed, particularly in feline cells or tissue. The basis of this is the successful cloning of the coded sequences of both subunits in recombinant expression constructs.

The p40 subunit can be cloned with procedures conventionally used by experts. The recombinant DNA and molecular biology techniques usefully employed in the broad practice of the present invention are more fully described in the List of References section hereof. An exact description of a possible procedure is given below in Example 1. This is recombination in the expression plasmid pMol (pMol-fIL12p40), which offers a way to produce minimalistic expression constructs.

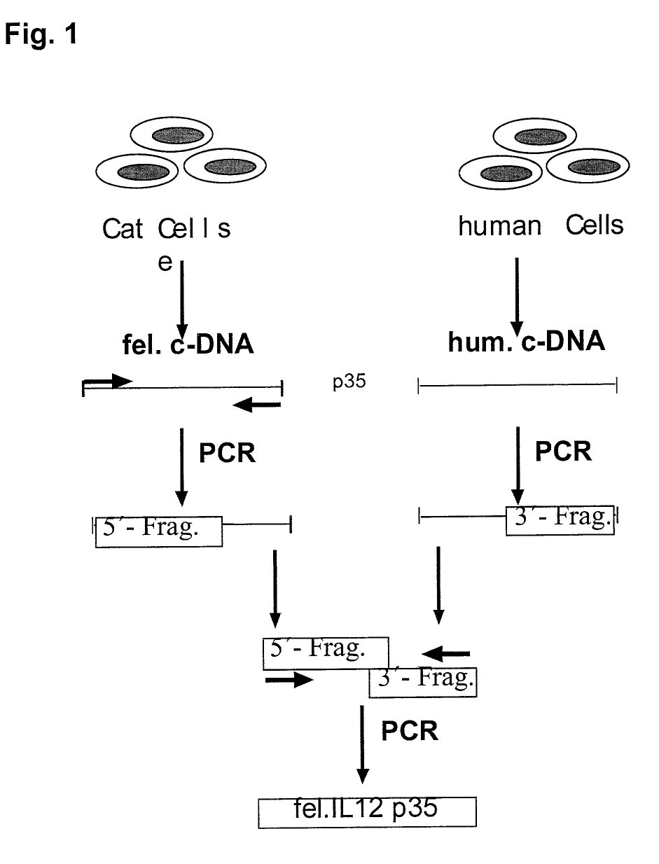

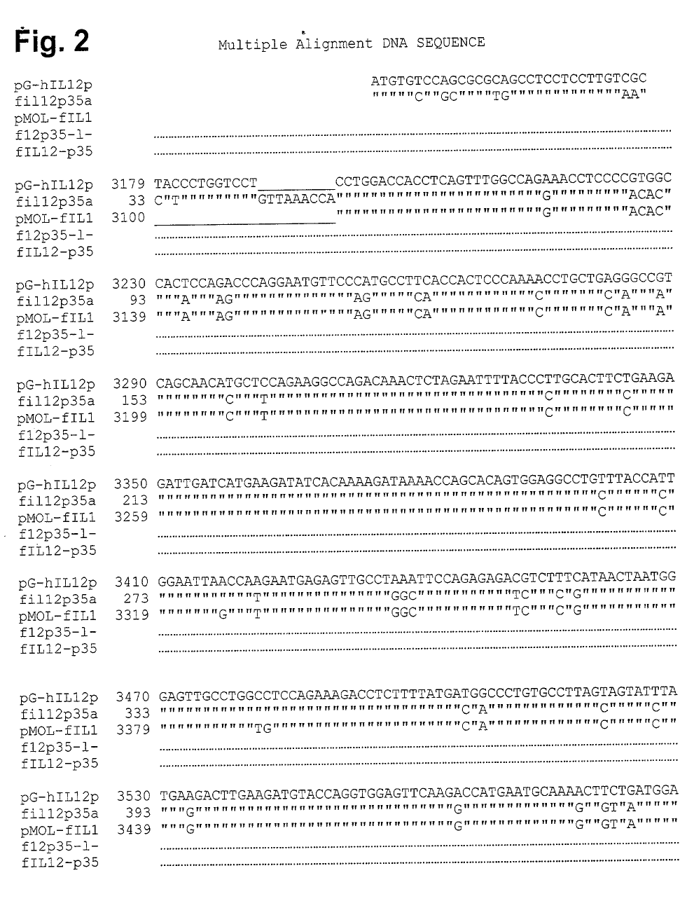

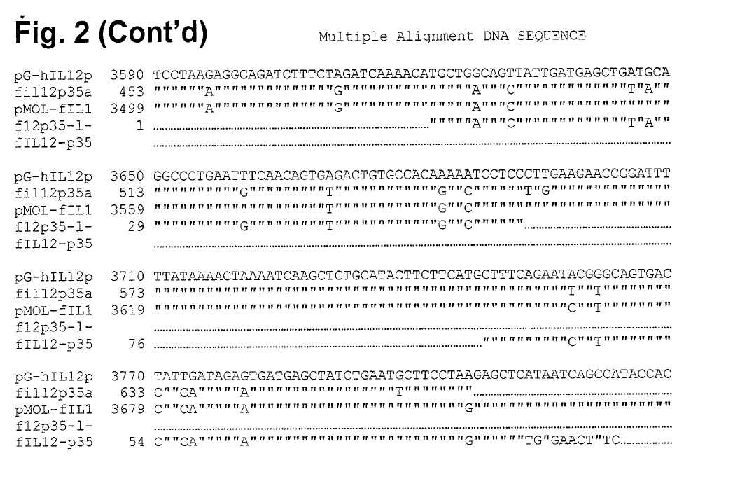

In contrast, the cloning of the expression construct of p35 turned out to be extremely difficult. The sequence which had been published earlier had been extracted from two overlapping clones. Despite multiple attempts by experienced scientists, it has not yet been possible to clone amplified cDNA for the full sequence length of the coding region of p35 from RNA-stimulated lymphocytes. The isolated recombinant sequences always had deletions in the 3′ region area of the coding region. A semi-synthetic strategy which exploited the great similarity between human p35 and feline IL-12 in the 3′ region was finally adopted. The 5′ region of the p35 sequence of feline cDNA could then be amplified. The 3′ region of the planned construct was amplified from a human sequence; in the primers used for this, some bases were chosen so that when there was a difference between the human and feline sequences, the feline sequence was chosen. The resulting 3′ construct overlapped with the amplified 5′ construct isolated from feline sequences. The two constructs were then heated to separate the strands and converted in a PCR reaction with primers, which bound to the ends of the total fragment. Astonishingly and contrary to expectation, no recombinants were found in which the primary sequences used in the PCR reaction were present at full length. Instead of this, bases which occur in the human sequences were repeatedly found within the primer sequences used. These point mutations led to a series of substitutions in the amino acid sequence of the p35 protein. The cause of this phenomenon is unclear. A sketch of the cloning strategy is presented in FIG. 1. A possible method for total cloning is given in Example 1. FIG. 2 contains a comparison of the sequence with the published sequence.

The previously outlined amplification of IL12-p35 led to the surprising result that an expressible functional p35 sequence had been inserted into the expression plasmid pMol (pMol-fIL12p35).

After restriction, ligation and digestion, linear, double-stranded expression constructs with covalently bound end groups were formed from the recombinants pMol-fIL12p35 and pMol-fIL12p40 (see Example 1 c). Unless otherwise stated, all further experiments were performed with these constructs..

Aside from the subunits of feline IL-12, which are shown in SEQ ID NO 1 and SEQ ID NO 2, other sequences, both polypeptide and polynucleotide, can be inserted, as long as they have at least about 95% homology (sequence similarity) with the above sequences. In the context of this invention, the homology was determined with the Complign program in the Mac Molly Tetra software package (www.mologen.com), with the following parameters: gap penalty=3; mismatch penalty=1.

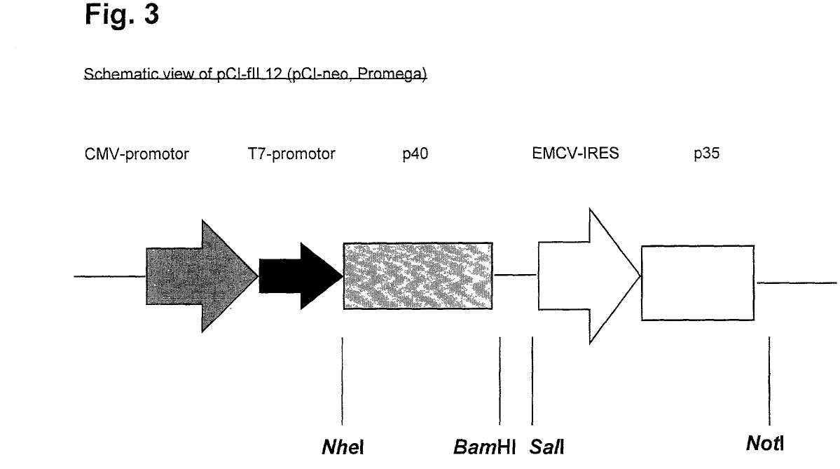

Active IL-12 can be synthesised, for example, by transfecting SP 2/0 cells with a DNA construct, which contains IL-12 under the control of only one promoter. This strategy helps to ensure that the two subunits are expressed at the same place and at the same time. One way of achieving this is to insert a so-called “internal ribosomal entry site” (IRES sequence) behind the sequence which codes for the smaller subunit (compare FIG. 3). The function can be proven by demonstrating the induction of γ-interferon (IFN) expression in vitro and in vivo after injection of the supernatant of transfected cells in cats. Control transfected cells do not exhibit this phenomenon. (For details see Examples 2a-2c).

A further experiment showed that transfection of cat cells with the genes coding for p35 and p40 led to these being able to stimulate cat lymphocytes kept in vitro in co-culture to produce γ-IFN. The aim of this experiment was to establish whether even separately administered genes are correctly translated and incorporated into functioning IL-12. To clarify whether this IL-12 is capable of achieving the desired biological effect, namely the induction of γ-interferon, the transfected lymphocytes were co-cultivated with lymphocytes from specific pathogen-free (SPF) cats. Aliquots were extracted at periodic intervals from the co-cultures and the γ-interferon mRNA was determined from these (see Example 3).

Within the framework of further studies, cats were immunised with a DNA vaccine which was specific for FIV, using IL-12 DNA as adjuvant. Parallel to this, cats were immunised with FIV-DNA alone. A schedule of three immunisations in intervals of three weeks proved to be very suitable. The degree of immunisation can be determined if, for example, three weeks after the last infection the vaccinated animals are given a test FIV infection together with unvaccinated animals. Periodic blood tests can be used to see how the immunisation is proceeding. In one experiment with four cats in each group, it was shown that the animals in Group 1 (vaccinated with FIV-DNA and IL-12) had better results with respect to all relevant parameters with which the infection can be characterised than the animals in Group 2 (vaccinated with FIV-DNA alone) (see Example 4).

Embodiments

### EXAMPLE 1a

### Recombinant Feline IL-12, p40

Peripheral feline blood lymphocytes were stimulated with Staphylococcus Protein A, RNA was isolated, cDNA produced and amplified under the standard reaction conditions of the polymerase chain reaction, using the primer pairs 5′-GAGAGTTCTC AGAGCTCCTA ACTGCAGGAC ACGGATG (SEQ ID NO 3) and 5′-GTAGCGGATA AGGTACCATG CATCCTCAGC AGTTGGT (SEQ ID NO 4). The amplified sample was introduced into the vector “Topo” (Invitrogen) and replicated in bacteria. After isolation of clones and checking the sequence, a recombinant was selected, the inserted and amplified p 40-coding sequence was excised from the p40-coding sequence by restriction with the enzymes KpnI and Sst1 and inserted into Vector pG (Mologen, Berlin). The sequence was confirmed and then amplified under standard PCR conditions with the primers 5′-GTAGCGGATA AGGTACCATG CATCCTCAGC AGTTGGT (SEQ ID NO 4) and 5′-GAGAGTTCTC AGAGCTCATC CTGGGGGTGG AACCTAA (SEQ ID NO 5). The isolated amplified sample was digested with the restriction endonucleases SstI und KpnI and inserted between the KpnI and SstI restriction sites in the vector pMol using standard methods. The result was the plasmid pMol-fIL12p40.

### EXAMPLE 1b

### Recombinant Feline IL-12, p35

Feline peripheral blood lymphocytes were stimulated with Staphylococcus Protein A, RNA isolated, cDNA formed and amplified by the polymerase chain reaction under standard conditions, using the primer pairs fIL12-p35(eco−)-r (76mer) 5′-GAGAGTTCTC AGAGCTCCTA GGAAGCATTC AGATAGCTCA TCATTCTATT GATGGTCACT GCACGGATTC TGAAAG (SEQ ID NO 6) and fil12-p35-l (37mer) 5′-GTAGCGGATA AGGTACCATG TGCCCGCCGC GTGGCCT (SEQ ID NO 7). The length of the amplified sample was shorter than expected. Human p35-coding plasmid pMOLhIL12p35 was inserted as template for amplification with the primers f12p35-l-lang (71 mer) 5′-TGCTGACAGC TATTGATGAG CTGTTACAGG CCCTGAATGT CAACAGTGTG ACTGTGCCAC AGAACTCCTC C (SEQ ID NO 8) and fIL12-p35(eco−)-r (76mer) 5′-GAGAGTTCTC AGAGCTCCTA GGMGCATTC AGATAGCTCA TCATTCTATT GATGGTCACT GCACGGATTC TGAAAG (SEQ ID NO 9) and amplified with polymerase chain reaction under standard conditions. The resulting amplified sample was isolated and further amplified with the amplified sample from Step 1 and the primers fil12-p35-l (37mer) and fIL12-p35(eco−)-r (76mer), using the polymerase chain reaction and standard reaction conditions. The resulting amplified sample was digested with the restriction endonucleases SstI and KpnI and inserted between the KpnI and SstI restriction sites of the pMol vector using standard methods. The result was the plasmid pMol-fIL12p35.

### EXAMPLE 1c

### Minimalistic Linear Covalently Closed Expression Construct

1 mg of the plasmid pMol-fil12p40 (for sequence see SEQ ID NO 10 in the summary of sequences, Sequence Protocol) was completely digested with the restriction endonuclease Eco31I . The resulting fragments were reacted overnight at 37° C. in 5 ml buffer with 50 μg of the 5′-phosphorylated desoxyoligoribonucleotide with the sequence AGGGGTCCAG TTTTCTGGAC (SEQ ID NO 11) (TIB Molbiol, Berlin), in the presence of 20 U T4 DNA-Ligase (MBI-Fermentas, Vilnius, Lithuania) and 10 U Eco31I. The reaction was stopped by heating to 60° C.

The resulting reaction mixture was concentrated, the buffer changed and digested overnight with 100 U restriction endonuclease HindIII and 100 U T7-DNA-polymerase in the absence of desoxyribonucleotides. The resulting product was purified by anionic ion-exchange chromatography, checked by gel electrophoresis and PCR and shown to be free of residues of the undesired fragment.

### EXAMPLE 2a

### In vitro Transcription/Translation of the Two IL-12 p35 and p40 Chains

The following experiment was carried out to check the function of the IRES-IL-12 construct. First, 3 constructs were assembled. Construct 1 was based on the p40 sequence produced by PCR which had been inserted into the pCI-neo vector (Promega). The plasmid contained the CMV and the T7 promoter and was named pCI-p40. Construct 2 was based on the p35 sequence produced by PCR which had been inserted into the pCITE4a(+) vector (Novagen). The plasmid contained the IRES (internal ribosomal entry site), which preceded the p35 sequence. This plasmid was named pCITE-p35. Construct 3 corresponded to the construct shown in FIG. 3 and was named pCI-fIL-12, In vitro translation was carried out with the above 3 constructs to check the correct translation of the two subunits p35 and p40. For this purpose, the plasmids pCI-p40, pCITE-p35 and pCI-fIL-12 were linearised and transcribed in vitro with the T7 Cab Scribe Kits (Boehringer Mannheim). The RNA was purified and used for in vitro translation with the Flexi™ Rabbit Reticulocyte Lysate Systems (Promega). The translation products were labelled with ³⁵S-methionine. After translation, the newly synthesised proteins were separated according to molecular weight on an SDS gel. The gel was dried in vacuum and exposed against a film, which was then developed. The bands observed on the film were of the expected molecular weights for p35 and p40 (Table 1).

TABLE 1
Results of the in vitro translation with the 3 constructs

| Vector | p40 | P35 | Comments |
|---|---|---|---|
| pCl-flL-12 | x | X | Bands corresponded to the expected |
| | | | molecular weights |
| pCl-p40 | x | — | Band corresponded to the expected |
| | | | molecular weight |
| pCITE-p35 | — | X | Band corresponded to the expected |
| | | | molecular weight |

It can be seen from these results that the construct pCI-fIL-12 is capable of correctly synthesising the two chains p35 and p40.

### EXAMPLE 2b

### In vitro Induction of γ-IFN in Cat Cells by Incubation with Cell Culture Supernatant which Contains IL-12

The next experiment was carried out to check the function of the IL-12 contruct (as in FIG. 3). First, the plasmid pCI-fIL-12 was linearised. The DNA was either transcribed in vitro using the T7 Cab Scribe Kits (Boehringer Mannheim) or used directly for transfection. The resulting RNA was used for the short term transfection of BHK-21 cells. The RNA transfection was carried out under routine conditions. Parallel to the transfection with the RNA derived from the plasmid, the BHK-21 cells were transfected with water under the same conditions. This served as the negative control. SP2/0 Cells were used for transfection with the DNA, followed by selection of the transfected cells and 24 h culture on G418 medium. Here too, cells were transfected with water as control. Non-transfected cells died within 7 days, as they were not protected from the toxic action of neomycin because they lacked the neomycin resistance gene. IL-12 was presumed to be present in the cells which had been transfected with RNA or DNA and supernatant from these cells was used for the culture of lymphocytes which had been freshly isolated from specific pathogen-free (SPF) cats. Before their treatment with the cell culture supernatants which contained IL-12, these lymphocytes had been incubated for 72 hours at 37° C. with 0.1% phytohaemagglutinin, so that they had the chance to produce the IL-12 receptor. After this, the lymphocytes were washed twice with sterile medium and then incubated for 48 hours with the cell supernatant which contained IL-12. After the culture, the cells were washed and their RNA extracted with Trizol Reagent (Gibco). With the random hexamer as primer, the RNA was subjected to reverse transcription, producing cDNA. The same quantities of the resulting cDNA were used for γ-IFN and the housekeeping gene GAPDH in the subsequent PCR amplification. PCR products were separated electrophoretically on 2% agarose gel, stained with ethidium bromide. The gels were then photographed and the fluorescence measured by densitometry. The colour intensity of γ-IFN relative to that of GAPDH was used for evaluation. As the GAPDH gene is always expressed to the same extent, it can be used for internal standardisation. Table 2 summarises the results, which were obtained with lymphocytes from 2 cats.

TABLE 2
Induction of γ-IFN in lymphocytes from 2 SPF cats.

| | Ratio of γ-IFN-mRNA | |
|---|---|---|
| | to GAPDH-RNA | |
| | | IL-12 Transf. |
| Supernatant used from: | Negative Control | Cells |
| BHK-21 Cells transfected with RNA | 0.0 | 0.310 |
| SP2/0 Cells transfected with DNA | 0.06 | 0.250 |

It is evident that the supernatant of cells which have been transfected with the IL-12 construct is capable of inducing γ-IFN, while the negative control cells cannot induce γ-IFN.

### EXAMPLE 2c

### In vivo Induction of γ-IFN in Cats by Injection of IL-12 Protein

To clarify whether the IL-12 produced in cell culture by transfection of SP2/0 cells with IL-12 DNA (pCI-fIL-12) was active in the cat, 2 cats were injected intramuscularly with aliquots of the cell culture supernatant which contained IL-12 or with the negative control culture supernatant. Blood was taken periodically from the cats, from which lymphocytes were extracted. RNA was extracted from the lymphocytes and subjected to reverse transcription. The resulting cDNA was then used for amplification and quantification of the γ-IFN and GAPDH sequences with the TaqMan Method (European Patent Application No. 98 124 317.3). The results are summarised in Table 3.

TABLE 3
Ratio of γ-IFN-mRNA to GAPDH-mRNA in lymphocytes from cats
which had previously been injected with cell culture supernatant.

| | Ratio of γ-IFN-mRNA to GAPDH-mRNA | | | | | | | |
|---|---|---|---|---|---|---|---|---|
| | 0 | 2 | 6 | 10 | 16 | 24 | 36 | 48 |
| Cat injected with: | Hours after Injection of the Supernatant | | | | | | | |
| Supernatant containing IL-12 | 0.0 | 0.2 | 0.2 | 0.1 | 0.3 | 6.5 | 1.2 | 0.3 |
| Control Supernatant without | 2.1 | 0.1 | 0.7 | 0.3 | 0.1 | 1.7 | 0.0 | 0.1 |
| IL-12 | | | | | | | | |

It can be seen from this experiment that injection of IL-12 in the cat leads to synthesis of γ-IFN by the cells of the lymphatic system 16 to 24 hours later.

### EXAMPLE 2d

### Investigation of the Function of Complete IL-12 with Respect to the Individual Chains p35 and p40 after Ballistic Transfer into the Feline Cell Line 3201.

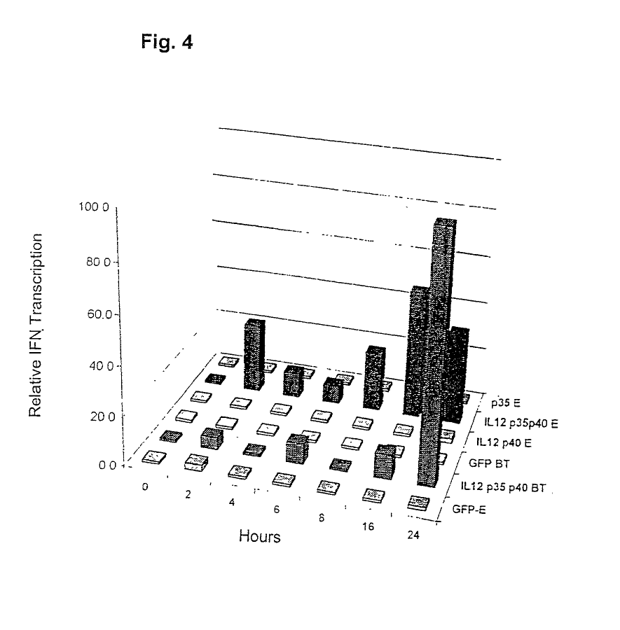

The method of ballistic transfection of target cells is described in documents WO91/00539 EP 500799. An apparatus for this purpose is disclosed in WO95/19799. 3201 cells were bombarded with small gold balls (diameter 1 μm). The small gold balls were previously coated with the gene coding for p35 and p40 or with the gene coding for the green fluorescing protein (GFP). Parallel to this, 3201 cells were also electrically transfected with the genes coding for p35 alone, for p40 alone, for both p35 and p40 and for GFP. Aliquots of the 3201 cells were then co-cultivated for 24 hours with SPF lymphocytes. The lymphocytes were periodically harvested and examined for the expression of γ-IFN, as described in Example 2c. The results are shown in FIG. 4. It is evident that transfection with either small gold balls or electric current leads to the production of γ-IFN 16 to 24 hours after co-cultivation of the transfected cells with lymphocytes. Ballistic or electrical transfection with either GFP or the p35 or p40 genes alone does not lead to synthesis of γ-IFN.

### EXAMPLE 3

### Immunisation of the Cat against FIV Using IL-12 as Adjuvant

An experiment was carried out to clarify whether IL-12 as adjuvant is capable of increasing the efficacy of a vaccine. Three groups of 4 cats each were used. The basic antigen in all groups, with the exception of the control group, was the gene which codes for the gp140-SU-Antigen. This is a gene construct which will be designated here as gp140-DNA. Vaccination by direct injection of naked DNA is disclosed in U.S. Pat. No. 5,580,859, U.S. Pat. No. 5,589,466 and U.S. Pat. No. 5,593,972. The DNA constructs were prepared as in Wittig et al. (WO 98/21322); they contained minimalistic expression constructs and consisted solely of the coding sequence, in front of which the sequence of the cytomegalovirus promoter (CMV) had been inserted. The coding sequence and the CMV promoter were used as linear double-stranded molecules, covalently closed at both ends, to prevent extra- or intracellular degradation by exonucleases. The DNA constructs were adsorbed on small gold particles, which were shot directly into the skin of the experimental animals. The animals were bombarded three times, at three week intervals, with the corresponding constructs. For each shot the DNA was mounted on 1 mg gold. A Helios gene-gun (Bio-rad, Munich, Germany; Bio-Rad Laboratories Headquarters at 1000 Alfred Nobel Drive, Hercules, Calif. 94547) and a pressure of 500 psi were used for immunisation. The total DNA dose came to approximately 2 μg per animal per vaccination. Four weeks after the third immunisation, the animals were given a test infection of an FIV strain (Zurich 2 Strain, (Morikawa et al., 1991)) which is used for isolating vaccine antigen. The dose used for the test infection was 25 times the concentration which would lead to infection in 50% of cats (cat infective dose 50=CID₅₀). The different groups were made up as follows:

TABLE 4
Composition of the vaccine groups

| Group | | |
|---|---|---|
| No. | Vaccine contains: | Issue |
| 1 | Only gold particles | Negative control, no protection expected |
| | | in these cats |
| 2 | gp140-DNA | Efficacy of the gp140 DNA construct |
| | | alone |
| 3 | gp140-DNA + | Clarification of the efficacy of IL-12 in |
| | IL-12-DNA | comparison with Group 2 |

The protective effect of the different vaccine preparations was examined at weekly intervals by measuring the following parameters (Exception: RNA loads were measured only in Week 5):

1. Antibodies to the transmembrane protein (TM) were measured with an ELISA test (Calzolari et al. 1995).

2. The quantity of FIV-RNA in the plasma of these cats was determined with a TaqMan®-PCR procedure.

3. The quantity of FIV-DNA, the so-called provirus-DNA, incorporated in the DNA of the lymphocytes was measured with a TaqMan® procedure (for a description of the TaqMan procedure see: Leutenegger et al. 1999).

The results summarised as follows:

1. Seroconversion against TM: The course of the seroconversion is summarised in Table 5.

2. It can be seen here that the animals in Control Group 1 seroconverted extraordinarily strongly, which suggests that the rate of virus replication is very high. From the fifth week all four animals were seropositive.

In Group 2 seroconversion developed only gradually and the degree of seroconversion was much lower than in Group 1. In Group 2 not all animals were seropositive even in the ninth week. This suggests reduced virus replication, which is compatible with protection.

In Group 3 only one animal had seroconverted up to the seventh week and the others remained fully negative. This suggests that complete protection had been achieved in three of the four animals. Comparison with the animals in Group 1 shows that the degree of seroconversion in the single positive animal was reduced, pointing to only moderate virus replication.

TABLE 5
IL-12 as adjuvant, FIV vaccine experiment, TM-ELISA results

| | | Weeks after Test Infection | | | | | | | | | | | | |
|---|---|---|---|---|---|---|---|---|---|---|---|---|---|---|
| Group | Cat | −7 | −5 | −3 | 0 | 1 | 2 | 3 | 4 | 5 | 6 | 7 | 8 | 10 |
| 1 | 2916 | 0.7 | 0 | 0 | 0 | 0.3 | 0.1 | 0 | 1.6 | 70.9 | 92.2 | 86.2 | 85.9 | 85.9 |
| | 2932 | 1.2 | 0.3 | 0.2 | 0 | 0.8 | 0.4 | 0 | 1.6 | 59.8 | 78.6 | 96.2 | 65.6 | 65.6 |
| | 381 | 0.6 | 1.3 | 0.1 | 0 | 0.4 | 0.2 | 0 | 5.3 | 78 | 95.3 | 72.6 | 89.3 | 89.3 |
| | 384 | 0 | 1 | 0.1 | 0 | 0.4 | 0.7 | 0 | 10.7 | 83.8 | 90.7 | 78.7 | 79.4 | 79.4 |
| 2 | 2924 | 0 | 0.3 | 0 | 0 | 0.04 | 0.8 | 0.4 | 1.9 | 76.4 | 94.3 | 88.7 | 88.3 | 88.3 |
| | 2947 | 0 | 1 | 0.5 | 0 | 0.4 | 0.6 | 0 | 0 | 0.4 | 1.3 | 0 | 60.4 | 60.4 |
| | 379 | 0.6 | 0.9 | 0.1 | 0 | 0 | 0.4 | 0 | 0.4 | 0.8 | 16.7 | 31.6 | 82.4 | 82.4 |
| | 393 | 0 | 0.3 | 0 | 0 | 0 | 0 | 0 | 0.8 | 66.7 | 85.7 | 63.6 | 90 | 90 |
| 3 | 2917 | 0 | 1.2 | 0.2 | 0 | 0 | 0.2 | 0 | 0 | 0.1 | 1.1 | 0 | 0.9 | 0.9 |
| | 2943 | 0 | 0.8 | 0 | 0 | 0 | 0.8 | 0 | 0 | 0 | 0.9 | 0 | 0.8 | 0.8 |
| | 377 | 1 | 2.4 | 2.3 | 0 | 2.6 | 0.4 | 0 | 1.6 | 0 | 2.1 | 0 | 0.5 | 0.5 |
| | 388 | 0 | 0.2 | 0 | 0 | 0.4 | 0.1 | 0 | 0 | 54.6 | 86.6 | 68.8 | 80.1 | 80.1 |

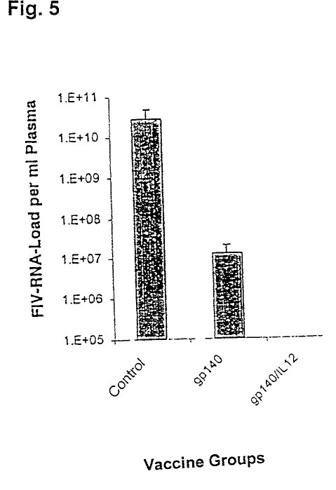

2. Plasma Load of FIV viral RNA: The results of the quantification of FIV-RNA in cat plasma are summarised in FIG. 5. The results can be commented on as follows:

Group 1: The load of viral RNA was highest here.

Group 2: The cats vaccinated with gp140-DNA exhibited significantly lower loads than the control animals, which suggests that the gp140 construct alone affords partial protection. These results are in accordance with the serology.

Group 3: Addition of IL-12 to the gp140-DNA provides complete protection against virus in the blood. These results also match the serology.

1. Quantity fo proviral DNA: The results of the quantification of proviral DNA in all cats are summarised in Table 6. The results may be commented on as follows:

Group 1: As with the serology and the RNA measurements, the animals in Group 1 were fully susceptible to the test reaction.

Group 2: The animals in Group 2 also became provirus positive without exception. The mean quantity of FIV provirus was only slightly less than in the control group.

Group 2: As previously found with the serology and quantity of RNA, 3 of the 4 cats were fully protected.

TABLE 6
Provirus load in the individual cats

| | | | Test | Week | | | | | | | |
|---|---|---|---|---|---|---|---|---|---|---|---|
| Group | Vaccine | Cat | Infect. | 1 | 2 | 3 | 4 | 5 | 6 | 8 | 10 |
| 1 | Gold | 2916 | 0.00 | 0.00 | 0.00 | 0.00 | 720.65 | 2036.35 | 3250.62 | 2150.45 | 617.83 |
| | | Y | | | | | | | | | |
| | | 2932 | 0.00 | 0.00 | 0.00 | 0.00 | 471.70 | 736.38 | 11649.83 | 490.65 | 570.69 |
| | | Y | | | | | | | | | |
| | | 0381 | 0.00 | 0.00 | 0.00 | 0.00 | 1674.35 | 5853.32 | 9818.22 | 1676.30 | 2080.08 |
| | | Y | | | | | | | | | |
| | | 0384 | 0.00 | 0.00 | 0.00 | 0.00 | 114.12 | 796.11 | 9393.60 | 9042.02 | 1570.48 |
| | | Y | | | | | | | | | |
| 2 | gp140 | 2924 | 0.00 | 0.00 | 0.00 | 0.00 | 1867.55 | 5395.20 | 6378.60 | 17906.93 | 2661.38 |
| | | Y | | | | | | | | | |
| | | 2947 | 0.00 | 0.00 | 0.00 | 528.48 | 526.56 | 0.00 | 8782.45 | 949.53 | 711.82 |
| | | Y | | | | | | | | | |
| | | 0379 | 0.00 | 0.00 | 0.00 | 0.00 | 3628.17 | 0.00 | 7096.10 | 34.97 | 205.24 |
| | | Y | | | | | | | | | |
| | | 0393 | 0.00 | 0.00 | 0.00 | 0.00 | 344.56 | 1470.99 | 6683.63 | 709.22 | 2449.24 |
| | | Y | | | | | | | | | |
| 3 | gp140 + | 2917 | 0.00 | 0.00 | 0.00 | 0.00 | 0.00 | 0.00 | 0.00 | 0.00 | 0.00 |
| | IL-12- | Y | | | | | | | | | |
| | DNA | 2943 | 0.00 | 0.00 | 0.00 | 0.00 | 0.00 | 0.00 | 0.00 | 0.00 | 0.00 |
| | | Y | | | | | | | | | |
| | | 0377 | 0.00 | 0.00 | 0.00 | 0.00 | 0.00 | 0.00 | 0.00 | 0.00 | 0.00 |
| | | Y | | | | | | | | | |
| | | 0388 | 0.00 | 0.00 | 0.00 | — | 4555.94 | 4644.27 | 451.29 | 429.79 | 263.25 |
| | | Y | | | | | | | | | |

In summary, it can be reported that using IL12 DNA together with gp140-DNA induces a better protective effect. This is manifested in a reduction in virus replication, which leads to less or no seroconversion and/or to less integration of viral DNA of the host cell.

One feature of an embodiment of the invention resides broadly in feline interleukin-12 fil12 polypeptide, which is expressed in eukaryotic or prokaryotic cells using the methods of recombinant gene expression in the form of the two polypeptide chains of the subunit p35 and p40 of feline interleukin 12, and where the corresponding proteins are prepared in such a way that they can be used in equimolar concentrations in the presence of a suitable antigen for immunisation of carnivores, specifically the domestic cat.

Another feature of an embodiment of the invention resides broadely in polypeptide, in which the subunit p35 of feline IL-12 is amplified with a plasmid coding for human IL-12 p35, which serves as template.

Yet another feature of an embodiment of the invention resides broadly in polypeptide, with at least 95% sequence homology to that of the polypeptide which is coded by the nucleotide sequence fIL12p40 SEQ ID NO 1 and fIL12p35 SEQ ID NO 2; its use as an immunostimulant, in particular for the prevention and treatment of disease in carnivores, specifically the domestic cat.

Still another feature of an embodiment of the invention resides broadly in nucleic acid construct coding for feline interleukin-12, which has sequences with at least 95% homology to the sequences of fIL12p40 SEQ ID NO 1 and fIL12p35 SEQ ID NO 2; its use as an immunostimulant for the immunisation against infectious diseases and/or the treatment of tumours and infectious diseases in Felidae, specifically the domestic cat.

A further feature of an embodiment of the invention resides broadly in nucleic acid construct, in which the sequences are controlled by a promoter and terminator sequence which is active in higher animals, such as carnivores, particularly Felidae, specifically the domestic cat.

Another feature of an embodiment of the invention resides broadly in nucleic acid construct, in which the construct consists of linear double-stranded DNA which is covalently bound at both ends and which has only one promoter and coding sequence per strand.

Yet another feature of an embodiment of the invention resides broadly in the use of the nucleic acid construct as adjuvant in prophylactic immunisation against viral diseases of carnivores, particularly Felidae, specifically the domestic cat.

Still another feature of an embodiment of the invention resides broadly in the use of the nucleic acid construct in accordance with claims 4 to 6 as treatment of diseases in which there is a deficiency in TH1, particularly when infection with FIV, FeLV or FCoV is present, and/or immunisation against infections with FIV, FeLV or FCoV infections.

A further feature of an embodiment of the invention resides broadly in the use of the polypeptide as therapeutic agent for tumours and autoimmune diseases of carnivores, specifically the domestic cat.

Another feature of an embodiment of the invention resides broadly in the use of the polypeptide as therapeutic agent when there is a deficiency in TH1, particularly when infection with FIV, FeLV or coronarvirus is present in carnivores, specifically in the domestic cat.

Yet another feature of an embodiment of the invention resides broadly in a vaccine or therapeutic agent which contains a polypeptide and at least one suitable carrier.

Another feature of an embodiment of the invention resides broadly in a vaccine or therapeutic agent which contains a nucleic acid construct and at least one suitable carrier.

A feature of an embodiment of the invention resides broadly in a feline interleukin-12 (fIL-12) polypeptide, obtained by cellular recombinant DNA expression in the form of polypeptide chains of the subunits p35 and p40 of feline interleukin 12.

Another feature of an embodiment of the invention resides broadly in a polypeptide obtained by eukaryotic cellular recombinant DNA expression.

Yet another feature of an embodiment of the invention resides broadly in a polypeptide obtained by prokaryotic cellular recombinant DNA expression.

Still another feature of an embodiment of the invention resides broadly in a polypeptide obtained by cellular recombinant DNA expression of pCI-fIL-12.

A further feature of an embodiment of the invention resides broadly in a polypeptide wherein p35 and p40 are in equimolar concentrations in relation to each other.

Another feature of an embodiment of the invention resides broadly in a polypeptide wherein the subunit p35 of feline IL-12 is amplified with a plasmid coding for human IL-12 p35.

Yet another feature of an embodiment of the invention resides broadly in a vaccine for treatment or prophylaxis of infectious disease associated with TH-1 deficiency in carnivores, said vaccine comprising fIL-12 and an antigen immunizingly effective against said infectious disease.

Still another feature of an embodiment of the invention resides broadly in a vaccine wherein said antigen comprises gp140.

A further feature of an embodiment of the invention resides broadly in a vaccine further including a carrier.

Another feature of an embodiment of the invention resides broadly in a method of treating or preventing a disease and/or tumor associated with TH-1 deficiency in a carnivore subject, comprising administering to the carnivore subject a vaccine comprising fIL-12.

Another feature of an embodiment of the invention resides broadly in a method wherein said carnivore subject is a Felidae subject.

Another feature of an embodiment of the invention resides broadly in a method wherein said carnivore subject is a domestic cat.

Another feature of an embodiment of the invention resides broadly in a method wherein said vaccine further comprises an antigen immunizingly effective against said disease

Another feature of an embodiment of the invention resides broadly in a method wherein said antigen comprises gp140.

Another feature of an embodiment of the invention resides broadly in a method wherein said disease comprises at least one disease selected from the group consisting of FIV, FeLV, and FcoV.

Another feature of an embodiment of the invention resides broadly in a method comprising treating or preventing tumor.

Another feature of an embodiment of the invention resides broadly in a method wherein said disease comprises an autoimmune disease.

Another feature of an embodiment of the invention resides broadly in a method of immunizing a feline subject against a disease or tumor associated with TH-1 deficiency, comprising immunizing said feline subject with a DNA vaccine comprising a nucleic acid construct coding for feline interleukin-12 (fIL-12).

Another feature of an embodiment of the invention resides broadly in a method wherein the nucleic acid construct has sequences with at least 95% homology to the sequences of fIL12p40 (SEQ ID NO 1) and fIL12p35 (SEQ ID NO 2).

Another feature of an embodiment of the invention resides broadly in a method wherein the DNA vaccine further comprises an adjuvant.

Another feature of an embodiment of the invention resides broadly in a method wherein the adjuvant comprises gp140.

Another feature of an embodiment of the invention resides broadly in a nucleic acid having sequences with at least 95% homology to the sequences of fIL12p40 (SEQ ID NO 1) and fIL12p35 (SEQ ID NO 2).

Another feature of an embodiment of the invention resides broadly in a polypeptide having at least 95% sequence homology to that of the polypeptide encoded by the nucleotide sequence fIL12p40 (SEQ ID NO 1) and fIL12p35 (SEQ ID NO 2).

Another feature of an embodiment of the invention resides broadly in a method of treatment or prophylaxis of TH-1 deficiency-related disease or tumor in a carnivore subject, comprising administering to the carnivore subject an immunostimulant composition comprising at least one of: (i) a nucleic acid construct having sequences with at least 95% homology to the sequences of fIL12p40 (SEQ ID NO 1) and fIL12p35 (SEQ ID NO 2), and (ii) a polypeptide having at least 95% sequence homology to that of the polypeptide which is coded by the nucleotide sequence fIL12p40 (SEQ ID NO 1) and fIL12p35 (SEQ ID NO 2).

Another feature of an embodiment of the invention resides broadly in a method wherein the carnivore subject is selected from Felidae.

Another feature of an embodiment of the invention resides broadly in a method wherein the carnivore subject is a domestic cat.

Another feature of an embodiment of the invention resides broadly in a method wherein the immunostimulant composition comprises (i) a nucleic acid construct having sequences with at least 95% homology to the sequences of fIL12p40 (SEQ ID NO 1) and fIL12p35 (SEQ ID NO 2).

Another feature of an embodiment of the invention resides broadly in a method wherein the immunostimulant composition comprises (ii) a polypeptide having at least 95% sequence homology to that of the polypeptide which is coded by the nucleotide sequence fIL12p40 (SEQ ID NO 1) and fIL12p35 (SEQ ID NO 2).

Another feature of an embodiment of the invention resides broadly in a method wherein the immunostimulant composition comprises (i) a nucleic acid construct having sequences with at least 95% homology to the sequences of fIL12p40 (SEQ ID NO 1) and fIL12p35 (SEQ ID NO 2) and (ii) a polypeptide having at least 95% sequence homology to that of the polypeptide which is coded by the nucleotide sequence fIL12p40 (SEQ ID NO 1) and fIL12p35 (SEQ ID NO 2).

Another feature of an embodiment of the invention resides broadly in a nucleic acid construct coding for feline interleukin-12 (fIL-12), which has sequences with at least 95% homology to the sequences of fIL12p40 (SEQ ID NO 1) and fIL12p35 (SEQ ID NO 2).

Another feature of an embodiment of the invention resides broadly in a nucleic acid construct, in which the sequences are controlled by a promoter and terminator sequence that is active in higher animals.

Another feature of an embodiment of the invention resides broadly in a nucleic acid construct, in which the sequences are controlled by a promoter and terminator sequence that is active in Felidae.

Another feature of an embodiment of the invention resides broadly in a nucleic acid construct, in which the sequences are controlled by a promoter and terminator sequence that is active in the domestic cat.

Another feature of an embodiment of the invention resides broadly in a nucleic acid construct, in which the construct consists of linear double-stranded DNA that is covalently bound at both ends and that has only one promoter and coding sequence per strand.

Another feature of an embodiment of the invention resides broadly in a method of forming a therapeutic composition for treatment or prophylaxis of a disease or tumor associated with TH-1 deficiency, comprising recombinantly expressing, in eukaryotic or prokaryotic cells, polypeptide chains of subunits p35 and p40 of feline interleukin 12 from a nucleic acid construct encoding same; extracting said polypeptide chains; and formulating said polypeptide chains in said therapeutic composition, wherein subunits p35 and p40 are in equimolar concentration with respect to one another.

Another feature of an embodiment of the invention resides broadly in a method further comprising formulating an antigen in said therapeutic composition.

Another feature of an embodiment of the invention resides broadly in a method wherein said antigen comprises gp140.

Another feature of an embodiment of the invention resides broadly in a recombinant construct selected from the group consisting of pMol-fIL12p35, pMol-fIL12p40, pCI-fIL-12, pCI-p40, and pCITE-p35.

Another feature of an embodiment of the invention resides broadly in a method of forming a nucleic acid construct for expression of fIL-12, comprising: amplifying the 5′ region of cDNA of feline IL-12 p35 and the 3′ region of cDNA of human IL-12 p35, with primers yielding 3′ constructs overlapped with amplified 5′ constructs; separating strands of the constructs and subjecting same to PCR reaction, to yield said nucleic acid construct as a PCR reaction product.

Another feature of an embodiment of the invention resides broadly in a method of treatment or prophylaxis of a TH-1 deficiency-related disease or tumor in a carnivore, comprising administering to the carnivore an immunostimulant composition comprising at least one therapeutic agent selected from the group consisting of (a) feline interleukin 12, (b) polypeptides homologous to feline interleukin 12 having corresponding therapeutic effect on said disease or tumor, and nucleic acid precursors of (a) and (b).

Another feature of an embodiment of the invention resides broadly in a method wherein said at least one therapeutic agent comprises a therapeutic agent selected from the group consisting of:

(i) nucleic acid constructs having sequences with at least 95% homology to sequences of fIL12p40 (SEQ ID NO 1) and fIL12p35 (SEQ ID NO 2),

(ii) polypeptides expressed from nucleic acid constructs (i),

(iii) polypeptides having at least 95% sequence homology to polypeptide coded by the nucleotide sequence fIL12p40 (SEQ ID NO 1) and fIL12p35 (SEQ ID NO 2), and

(iv) nucleic acid constructs encoding polypeptides (iii).

Another feature of an embodiment of the invention resides broadly in a method wherein the carnivore is selected from the group consisting of Felidae.

Another feature of an embodiment of the invention resides broadly in a method wherein the Felidae carnivore is a domestic cat.

Another feature of an embodiment of the invention resides broadly in a method wherein TH-1 deficiency-related disease comprises a disease selected from the group consisting of FIV, FeLV, and FcoV.

Another feature of an embodiment of the invention resides broadly in a method wherein said at least one therapeutic agent comprises a therapeutic agent selected from the group consisting of:

(v) nucleic acid constructs having sequences with at least 95% homology to sequences of fIL12p40 (SEQ ID NO 1) and fIL12p35 (SEQ ID NO 2), and

(vi) polypeptides expressed from nucleic acid constructs (i).

Another feature of an embodiment of the invention resides broadly in a method wherein said at least one therapeutic agent comprises a polypeptide obtained by eukaryotic or prokaryotic cellular recombinant DNA expression.

Another feature of an embodiment of the invention resides broadly in a method wherein said cellular recombinant DNA expression comprises recombinantly expressing polypeptide chains of subunits p35 and p40 of feline interleukin 12 from nucleic acid encoding same, to produce said polypeptide.

Another feature of an embodiment of the invention resides broadly in a method wherein subunits p35 and p40 are in equimolar concentration with respect to one another in said immunostimulant composition.

Another feature of an embodiment of the invention resides broadly in a method wherein said cellular recombinant DNA expression includes amplification of subunit p35 of feline IL-12 with a plasmid coding for human IL-12 p35.

Another feature of an embodiment of the invention resides broadly in a method wherein said nucleic acid comprises a nucleic acid construct from the group consisting of pMol-fIL12p35, pMol-fIL12p40, pCI-fIL-12, pCI-p40, and pCITE-p35.

Another feature of an embodiment of the invention resides broadly in a method wherein said immunostimulant composition comprises at least one antigen.

Another feature of an embodiment of the invention resides broadly in a method wherein said at least one antigen comprises gp140.

Another feature of an embodiment of the invention resides broadly in a method of making a therapeutic composition for treatment or prophylaxis of a disease or tumor associated with TH-1 deficiency, comprising recombinantly expressing, in eukaryotic or prokaryotic cells, polypeptide comprising polypeptide chains of subunits p35 and p40 of feline interleukin 12 from nucleic acid encoding same; extracting said polypeptide; and formulating said polypeptide in said therapeutic composition, wherein subunits p35 and p40 are in equimolar concentration with respect to one another.

Another feature of an embodiment of the invention resides broadly in a method wherein said nucleic acid is formed by steps including: amplifying the 5′ region of cDNA of feline IL-12 p35 and the 3′ region of cDNA of human IL-12 p35, with primers yielding 3′ constructs overlapped with amplified 5′ constructs; separating strands of the constructs and subjecting same to PCR reaction, to yield said nucleic acid as a PCR reaction product.

Another feature of an embodiment of the invention resides broadly in a method wherein said nucleic acid is selected from the group consisting of nucleic acids having sequences with at least 95% homology to sequences of fIL12p40 (SEQ ID NO 1) and fIL12p35 (SEQ ID NO 2).

Another feature of an embodiment of the invention resides broadly in a method wherein said sequences with at least 95% homology to sequences of fIL12p40 (SEQ ID NO 1) and fIL12p35 (SEQ ID NO 2) are controlled by a promoter and terminator sequence that is active in Felidae.

Another feature of an embodiment of the invention resides broadly in a method further comprising incorporating at least one antigen in said therapeutic composition.

Another feature of an embodiment of the invention resides broadly in a method wherein said antigen comprises gp140.

Another feature of an embodiment of the invention resides broadly in a method wherein said nucleic acid comprises nucleic acid construct pCI-fIL-12.

The components disclosed in the various publications, disclosed or incorporated by reference herein, may be used in the embodiments of the present invention, as well as equivalents thereof.

The appended drawings in their entirety, including all dimensions, proportions and/or shapes in at least one embodiment of the invention, are accurate and are hereby included by reference into this specification.

All, or substantially all, of the components and methods of the various embodiments may be used with at least one embodiment or all of the embodiments, if more than one embodiment is described herein.

All of the patents, patent applications and publications recited herein, and in the Declaration attached hereto, are hereby incorporated by reference as if set forth in their entirety herein.

The following patent publications are incorporated by reference as if set forth in their entirety herein: U.S. Pat. No. 5,571,515, issued Nov. 5, 1996 to Phillip, et al. and European Patent No. 0 919 241 issued on Jun. 2, 1999 to Toray Industries.

The corresponding foreign and international patent publication applications, namely, Swiss Patent Application No. 1259/99, filed on Jul. 8, 1999, and International Application No. PCT/DE00/02263, filed on Jul. 8, 2000, having inventors Hans LUTZ, Christian LEUTNEGGER, Nils PEDERSEN, Matthias SCHROFF, and Burghardt WITTIG, as well as their published equivalents, and other equivalents or corresponding applications, if any, in corresponding cases in Switzerland and elsewhere, and the references and documents cited in any of the documents cited herein, such as the patents, patent applications and publications, are hereby incorporated by reference as if set forth in their entirety herein.

All of the references and documents, cited in any of the documents cited herein, are hereby incorporated by reference as if set forth in their entirety herein. All of the documents cited herein, referred to in the immediately preceding sentence, include all of the patents, patent applications and publications cited anywhere in the present application.

The details in the patents, patent applications and publications may be considered to be incorporable, at applicant's option, into the claims during prosecution as further limitations in the claims to patentably distinguish any amended claims from any applied prior art.

Some examples of devices and methods for the injection of DNA into a mammal which may possibly be used in a possible embodiment of the present invention may possibly be found in the following U.S. patents, which are incorporated by reference herein: U.S. Pat. Nos. 5,580,859, entitled “Delivery of exogenous DNA sequences in a mammal”; 5,589,466, entitled “Induction of a protective immune response in a mammal by injecting a DNA sequence”; 5,593,972, entitled “Genetic immunization”; 6,319,224, entitled “Intradermal injection system for injecting DNA-based injectables into humans”; 6,214,804, entitled “Induction of a protective immune response in a mammal by injecting a DNA sequence”; and 5,656,610, entitled “Producing a protein in a mammal by injection of a DNA-sequence into the tongue.”

Some examples of immunostimulants and uses thereof may be found in the following U.S. Pat. Nos. 5,336,666, entitled “Immunostimulant drug based on polar glyopeptidolipids of mycobacterium chelonae”, 5,250,296, entitled “Immunostimulant agent containing interleukin-2 and 5′-deoxy-5-fluorouridine”; 5,073,630, entitled “Polymeric anhydride of magnesium and proteic ammonium phospholinoleate with antiviral, antineoplastic and immunostimulant properties”; 5,041,535, entitled “Antileukemic and immunostimulant peptides”; 4,937,327, entitled “Derivative of D.25, process for its preparation, its use as an immunostimulant, and pharmaceutical compositions containing the derivative”; 4,910,296, entitled “Medicaments containing alpha 1 thymosin fragments and having an immunostimulant action, and fragments of alpha 1 thymosin”; 4,801,578, entitled “Muramylpeptide-glycoprotein immunostimulant derivatives, their preparation and their use in medication”; 4,737,521, entitled “Suramin sodium for use as an immunostimulant”; 4,501,693, entitled “Method of preparing immunostimulant proteoglycans which induce production of interferon, proteoglycans obtained and pharmaceutical compositions containing them”; 4,470,926, entitled “Medicaments containing thymosin alpha 1 fragments and having an immunostimulant action, and fragments of thymosin alpha 1”; 4,407,825, entitled “Novel bis- and poly-disulfides having immunostimulant activity”; 4,397,848, entitled “N-Substituted aziridine-2-carboxylic acid immunostimulant derivatives”; 4,376,731, entitled “1-Aziridine carboxylic acid derivatives with immunostimulant activity”; 4,337,243, entitled “Immunostimulant medicament and process of preparing same”; 4,285,930, entitled “Antigens comprising immunostimulant adjuvants and their use in immunotherapy”; 4,182,751, entitled “New immunostimulant medicament and process of preparing same”; 4,180,563, entitled “Immunostimulant agent from Salmonella typhimurium or Listeria monocytogenes bacterial cells and pharmaceutical composition”; 4,148,885, entitled “Immunostimulant medicine”; and 4,076,801, entitled “Immunostimulant agent, compositions thereof and methods for their preparation”.

Some examples of interleukin-12 and uses thereof which may possibly be used in a possible embodiment of the present invention may be found in the following U.S. Pat. Nos. 6,333,038, entitled “Prophylaxis of allergic disease”; 6,323,334, entitled “Nucleic acid molecules encoding a 103 gene product and uses therefor”; 6,316,420, entitled “DNA cytokine vaccines and use of same for protective immunity against multiple sclerosis”; 6,303,756, entitled “Tumor associated nucleic acids and uses therefor”; 6,288,218, entitled “Compositions and methods for the treatment and diagnosis of immune disorders”; 6,245,525, entitled “Tumor associated nucleic acids and uses therefor”; 6,239,116, entitled “Immunostimulatory nucleic acid molecules”; 6,207,646, entitled “Immunostimulatory nucleic acid molecules”; 6,204,371, entitled “Compositions and methods for the treatment and diagnosis of immune disorders”; 6,197,524, entitled “Methods for detecting, identifying, isolating, and selectively labelling and targeting TH1 lymphocyte by means of the LAG-3 protein”; 6,183,951, entitled “Methods of diagnosing clinical subtypes of crohn's disease with characteristic responsiveness to anti-Th1 cytokine therapy”; 6,174,527, entitled “Methods and compositions for gene therapy for the treatment of defects in lipoprotein metabolism”; 6,156,887, entitled “Compositions and methods for the treatment and diagnosis of immune disorders”; 6,150,502, entitled “Polypeptides expressed in skin cells”; 6,086,876, entitled “Methods and compositions for the inhibition of interleukin-12 production”; 6,084,083, entitled “Compositions and methods for the treatment and diagnosis of immune disorders”; 6,080,399, entitled “Vaccine adjuvants for immunotherapy of melanoma”; 6,066,498, entitled “Compositions for the treatment and diagnosis of immune disorders”; 48 6,066,322, entitled “Methods for the treatment of immune disorders”; 49 6,056,964, entitled “Immunotherapeutic agent and its use”; 5,980,898, entitled “Adjuvant for transcutaneous immunization”; 5,910,306, entitled “Transdermal delivery system for antigen”; 5,879,687, entitled “Methods for enhancement of protective immune responses”; and 5,876,735, entitled “Methods for enhancement of protective immune responses.”

Some examples of prophylaxis or treatment relating to TH1 which may possibly be used in a possible embodiment of the present invention may possibly be found in the following U.S. Pat. Nos. 6,333,325, entitled “Method of treating cytokine mediated diseases or conditions”; 6,333,038, entitled “Prophylaxis of allergic disease”; 6,331,299, entitled “Method for treatment of cancer and infectious disease and compositions useful in same”; 6,329,512, entitled “Immunogenic conjugate molecules”; 6,329,505, entitled “Compositions and methods for therapy and diagnosis of prostate cancer”; 6,328,978, entitled “Methods for the treatment of immunologically-mediated skin disorders”; 6,316,420, entitled “DNA cytokine vaccines and use of same for protective immunity against multiple sclerosis”; 6,309,847, entitled “Method for detecting or monitoring the effectiveness of treatment of T cell mediated diseases”; 6,303,756, entitled “Tumor associated nucleic acids and uses therefor”; 6,303,114, entitled “IL-12 enhancement of immune responses to T-independent antigens”; 6,261,281, entitled “Method for genetic immunization and introduction of molecules into skeletal muscle and immune cells”; 6,258,359, entitled “Immunogenic compositions against helicobacter infection, polypeptides for use in the compositions, and nucleic acid sequences encoding said polypeptides”; 6,248,330, entitled “Immunogenic compositions against helicobacter infection, polypeptides for use in the compositions, and nucleic acid sequences encoding said polypeptides”; 6,242,427, entitled “Methods of inhibiting phagocytosis”; 6,228,656, entitled “Method of cleaving specific nucleic acid sequence”; and 6,191,114, entitled “Immunological activity for a peptide of the limulus anti-LPS factor.”

Some examples of “gene-guns”and uses therefore which may possibly be used in a possible embodiment of the present invention may possibly be found in the following U.S. Pat. Nos. 6,322,780, entitled “Marek's disease virus vaccines for protection against Marek's disease”; 6,312,907, entitled “DbpA compositions and methods of use”; 6,306,832, entitled “Peptide antiestrogen compositions and methods for treating breast cancer”; 6,288,214, entitled “Collagen binding protein compositions and methods of use”; 6,258,788, entitled “DNA vaccines against tick-borne flaviviruses”; 6,255,289, entitled “Gene delivery by secretory gland expression”; 6,248,720, entitled “Method for gene therapy using nucleic acid loaded polymeric microparticles”; 6,248,517, entitled “Decorin binding protein compositions and methods of use”; 6,235,290, entitled “DNA immunization against chlaymdia infection”; 6,228,835, entitled “Decorin binding protein compositions”; 6,214,804, entitled “Induction of a protective immune response in a mammal by injecting a DNA sequence”; 6,214,355, entitled “DbpA compositions”; 6,207,400, entitled “Non- or minimally invasive monitoring methods using particle delivery methods”; 6,200,959, entitled “Genetic induction of anti-viral immune response and genetic vaccine for filovirus”; 6,183,746, entitled “Immunogenic peptides from the HPV E7 protein”; 6,180,614, entitled “DNA based vaccination of fish”; 6,143,211, entitled “Process for preparing microparticles through phase inversion phenomena”; 6,090,791, entitled “Method for inducing mucosal immunity”; 6,090,790, entitled “Gene delivery by microneedle injection”; 6,086,891, entitled “Bi-functional plasmid that can act as both a DNA vaccine and a recombinant virus vector”; 6,070,126, entitled “Immunobiologically-active linear peptides and method of identification”; 6,060,457, entitled “DNA plasmid vaccine for immunization of animals against BVDV”; 6,033,877, entitled “Peptide expression and delivery system”; 6,025,164, entitled “Bacterial antigens and vaccine compositions”; 6,020,192, entitled “Humanized green fluorescent protein genes and methods”; 6,020,154, entitled “H. influenzae HxuB and HxuC genes, proteins and methods of use”; 6,013,832, entitled “Process for the production of benzene derivatives”; 6,013,258, entitled “Immunogenic peptides from the HPV E7 protein”; 6,004,944, entitled “Protein delivery by secretory gland expression”; 5,989,553, entitled “Expression library immunization”; 1 5,972,657, entitled “Gene encoding outer membrane protein B1 of moraxella catarrhalis”; 5,968,750, entitled “Humanized green fluorescent protein genes and methods”; 5,965,139, entitled “Chicken infectious anemia virus vaccine”; 5,948,412, entitled “Vaccine for Moraxella catarrhalis”; 5,916,879, entitled “DNA transcription unit vaccines that protect against avian influenza viruses and methods of use thereof”; 5,885,971, entitled “Gene therapy by secretory gland expression”; 5,880,103, entitled “Immunomodulatory peptides”; 5,874,304, entitled “Humanized green fluorescent protein genes and methods”; 5,871,723, entitled “CXC chemokines as regulators of angiogenesis”; and 5,853,987, entitled “Decorin binding protein compositions and methods of use.”

Some examples of DNA and uses thereof for immunizations and vaccinations which may possibly be used in a possible embodiment of the present invention may possibly be found in the following U.S. Pat. Nos. 6,316,420, entitled “DNA cytokine vaccines and use of same for protective immunity against multiple sclerosis”; 6,316,004, entitled “Chimeric somatostatin containing protein and encoding DNA, plasmids of expression, method for preparing chimeric protein, strain-producers, immunogenic composition, method for increasing the productivity of farm animals”; 6,310,196, entitled “DNA construct for immunization or gene therapy”; 6,270,795, entitled “Method of making microencapsulated DNA for vaccination and gene therapy”; 6,262,172, entitled “Method for preparing a carbonized resin DNA immunoadsorbent”; 6,261,762, entitled “Cloned DNA sequences related to the entire genomic RNA of human immunodeficiency virus II (HIV-2), polypeptides encoded by these DNA sequences and the use of these DNA clones polypeptides in diagnostic kits”; 6,258,788, entitled “DNA vaccines against tick-borne flaviviruses”; 6,254,869, entitled “Cryptopain vaccines, antibodies, proteins, peptides, DNA and RNA for prophylaxis, treatment and diagnosis and for detection of cryptosporidium species”; 6,248,582, entitled “Gene deleted recombinant FeLV proviral DNA for production of vaccines against FeLV”; 6,238,669, entitled “Proteins encoded by chicken anemia virus DNA and diagnostic kits and vaccines employing said proteins”; 6,235,523, entitled “Vectors for DNA immunization against cervical cancer”; 6,235,290, entitled “DNA immunization against chlaymdia infection”; 6,231,863, entitled “DNA sequences, molecules, vectors and vaccines for feline calicivirus disease and methods for producing and using same”; 6,228,371, entitled “Mycobacterium tuberculosis DNA sequences encoding immunostimulatory peptides”; 6,225,292, entitled “Inhibitors of DNA immunostimulatory sequence activity”; 6,221,882, entitled “Methods for inhibiting immunostimulatory DNA associated responses”; 6,221,664, entitled “Composite vaccine which contains antigen, antibody and recombinant DNA and its preparing method”; 6,214,804, entitled “Induction of a protective immune response in a mammal by injecting a DNA sequence”; 6,194,389, entitled “Particle-mediated bombardment of DNA sequences into tissue to induce an immune response”; 6,187,759, entitled “Canine parvovirus DNA vaccination”; 6,187,320, entitled “Equine herpesviruses (EHV) which contain foreign DNA, process for the preparation thereof and the use thereof in vaccines”; 6,183,986, entitled “OspA DNA and lyme disease vaccine”; 6,180,614, entitled “DNA based vaccination of fish”; 6,165,993, entitled “DNA vaccines against rotavirus infections”; 6,159,751, entitled “Development of DNA probes and immunological reagents of human tumor associated antigens”; 6,110,898, entitled “DNA vaccines for eliciting a mucosal immune response”; 6,096,878, entitled “Human immunoglobulin V.sub.H gene segments and DNA fragments containing the same”; 6,086,891, entitled “Bi-functional plasmid that can act as both a DNA vaccine and a recombinant virus vector”; 6,083,689, entitled “Sensitive immunoassays utilizing antibody conjugates with replicable DNA templates”; 6,074,866, entitled “Shuttle vectors for the introduction of DNA into mycobacteria and utilization of such bacteria as vaccines”; 6,066,503, entitled “Recombinant DNA molecules encoding aminopeptidase enzymes and their use in the preparation of vaccines against helminth infections”; 6,063,385, entitled “DNA vaccine for parvovirus”; 6,060,457, entitled “DNA plasmid vaccine for immunization of animals against BVDV”; 6,004,799, entitled “Recombinant live feline immunodeficiency virus and proviral DNA vaccines”; 5,997,869, entitled “Peptides containing a fusion joint of a chimeric protein encoded by DNA spanning a tumor-associated chromosomal translocation and their use as immunogens”; 5,980,900, entitled “Amino acid DNA sequences related to genomic RNA of human immunodeficiency virus (HIV-1)”; 5,962,268, entitled “DNA encoding an immune cell cytokine”; 5,958,895, entitled “DNA vaccines for herpes simplex virus”; 5,939,400, entitled “DNA vaccination for induction of suppressive T cell response”; 5,916,879, entitled “DNA transcription unit vaccines that protect against avian influenza viruses and methods of use thereof”; 5,889,172, entitled “DNA sequences for immunologically active peptides of pertussis toxin”; 5,846,949, entitled “Method for eliciting an immune response using a gene expression system that co-delivers an RNA polymerase with DNA”; 5,843,937, entitled “DNA-binding indole derivatives, their prodrugs and immunoconjugates as anticancer agents”; 5,834,305, entitled “Attenuated herpesvirus, herpesvirus which include foreign DNA encoding an amino acid sequence and vaccines containing same”; 5,830,688, entitled “DNA sequences, vectors, recombinant viruses and method which employs recombinant vaccinia viruses capable of muliplying in CHO cells”; 5,795,872, entitled “DNA construct for immunization”; 5,788,962, entitled “DNA sequences coding for mycoplasma hyopneumoniae surface antigens, corresponding proteins and use in vaccines and diagnostic procedures”; 5,780,448, entitled “DNA-based vaccination of fish”; 5,780,289, entitled “Coccidiosis poultry vaccine DNA encoding an elmeria 20K antigen”; and 5,773,602, entitled “DNA fragments obtained from a novel human immunodeficiency virus designated LAV.sub.MAL.”

Some examples of IL-12 and uses thereof which may possibly be used in a possible embodiment of the present invention may possibly be found in the following U.S. Pat. Nos. 6,303,114, entitled “IL-12 enhancement of immune responses to T-independent antigens”; 6,225,117, entitled “Antibodies against human IL-12”; 6,168,923, entitled “Compositions and methods for use of IL-12 as an adjuvant”; 6,046,012, entitled “Antibody to IL-12 receptor”; 5,985,264, entitled “IL-12 Stimulation of Neonatal immunity”; 5,976,539, entitled “Compositions and methods for use of IL-12 as an adjuvant”; 5,928,636, entitled “Use of IL-12 and IFN.alpha. for the treatment of infectious diseases”; 5,922,685, entitled “IL-12 gene therapy of tumors”; 5,919,903, entitled “Low affinity human IL-12 beta2 receptor”; 5,891,680, entitled “Bioactive fusion proteins comprising the p35 and p40 subunits of IL-12”; 5,876,966, entitled “Compounds and methods for the stimulation and enhancement of protective immune responses and IL-12 production”; 5,853,714, entitled “Method for purification of IL-12”; 5,853,697, entitled “Methods of treating established colitis using antibodies against IL-12”; 5,840,530, entitled “DNA encoding receptors for the beta-2 chain of human IL-12”; 5,744,132, entitled “Formulations for IL-12”; 5,723,127, entitled “Compositions and methods for use of IL-12 as an adjuvant”; 5,665,347, entitled “IL-12 inhibition of B1 cell activity”; 5,571,515, entitled “Compositions and methods for use of IL-12 as an adjuvant.”

The invention as described hereinabove in the context of the preferred embodiments is not to be taken as limited to all of the provided details thereof, since modifications and variations thereof may be made without departing from the spirit and scope of the invention.

### List of References

The following references are incorporated by reference as if set forth in their entirety herein.

Baumberg, ed. Prokaryotic Gene Expression (Frontiers in Molecular Biology), Oxford Univ Press [1999].

Bush K. Day N K. Kraus L A. Good R A. Bradley W G. (1994) Molecular cloning of feline interleukin 12 p35 reveals the conservation of leucine-zipper motifs present in human and murine IL-12 p35. Molecular Immunology. 31(17):1373-4.

Calzolari M., Young E., Cox D., Davis D., Lutz H. Serological diagnosis of feline immunodeficiency virus infection using recombinant transmembrane glycoprotein, Vet.Immunol.Immunopathol. 46, 83-92 (1995)

Fehr, D., Dean, G. A., Huder, J., Fan, Z., Huettner, S., Higgins, J. W., Pedersen, N. C. and Lutz, H. (1997) Nucleotide and predicted peptide sequence of feline interleukin-12 (IL-12). DNA Sequence 8(1-2), 77-82.

Gately, M. K. and Mulqueen, M. J. (1996) Interleukin-12: potential clinical applications in the treatment and prevention of infectious diseases. [Review] [49 refs]. Drugs 52(Suppl 2), 18-25; discussion 25-6.

Glover, D. M. (ed.), 1985, DNA Cloning: A Practical Approach, MRL Press, Ltd., Oxford, U.K., Vol. I, II.

Guide to Molecular Cloning Techniques, Academic Press, Inc., San Diego, Calif., [1987].

Leutenegger C., Klein D., Hofmann-Lehmann R., Mislin C., Hummel U., Böni J., Boretti F., Guenzburg W., Lutz H; Rapid feline immunodeficiency virus provirus quantitation by polymerase chain reaction using the TaqMan® fluorogenic real-time detection system; Journal of Virological Methods 78, 105-116 (1999).

Mosmann, T. R., Cherwinski, H., Bond, M. W., Giedlin, M. A. and Coffman, R. L. (1986) Two types of murine helper T cell clone. I. Definition according to profiles of lymphokine activities and secreted proteins. Journal of Immunology 136(7), 2348-57.

Morikawa, S., Lutz, H., Aubert, A. and Bishop, D. H. (1991) Identification of conserved and variable regions in the envelope glycoprotein sequences of two feline immunodeficiency viruses isolated in Zurich, Switzerland. Virus Research 21(1), 53-63.

Piccotti J R. Chan S Y. Li K. Eichwald E J. Bishop D K. (1997) Differential effects of IL-12 receptor blockade with IL-12 p40 homodimer on the induction of CD4+ and CD8+ IFN-gamma-producing cells. Journal of Immunology. 158(2):643-8.

Recombinant DNA Laboratory Manual, Academic Press, Inc., San Diego Calif., [1999].

Sambrook et al., 1989, Molecular Cloning, A Laboratory Manual, Cold Spring Harbor Laboratory, 2d Ed., Cold Spring Harbor, N.Y.

Schijns, V. E., Wierda, C. M., Vahlenkamp, T. W. and Horzinek, M. C. (1997) Molecular cloning of cat interleukin-12. Immunogenetics 45(6), 462-3.

What is claimed is:

1. A method of treatment or prophylaxis of a TH-1 deficiency-related disease or tumor in a carnivore, comprising administering to the carnivore an immunostimulant composition comprising at least one therapeutic agent selected from the group consisting of (a) feline interleukin 12, (b) polypeptides homologous to feline interleukin 12 having corresponding therapeutic effect on said disease or tumor, and nucleic acid precursors of (a) and (b).

2. The method of claim 1, wherein said at least one therapeutic agent comprises a therapeutic agent selected from the group consisting of:
(i) nucleic acid constructs having sequences with at least 95% homology to sequences of fIL12p40 (SEQ ID NO 1) and fIL12p35 (SEQ ID NO 2),
(ii) polypeptides expressed from nucleic acid constructs (i),
(iii) polypeptides having at least 95% sequence homology to polypeptide coded by the nucleotide sequence fIL12p40 (SEQ ID NO 1) and fIL12p35 (SEQ ID NO 2), and
(iv) nucleic acid constructs encoding polypeptides (iii).

3. The method of claim 2, wherein the carnivore is selected from the group consisting of Felidae.

4. The method of claim 3, wherein the Felidae carnivore is a domestic cat.

5. The method of claim 4, wherein TH-1 deficiency-related disease comprises a disease selected from the group consisting of FIV, FeLV, and FcoV.

6. The method of claim 5, wherein said at least one therapeutic agent comprises a therapeutic agent selected from the group consisting of:
(i) nucleic acid constructs having sequences with at least 95% homology to sequences of fIL12p40 (SEQ ID NO 1) and fIL12p35 (SEQ ID NO 2), and
(ii) polypeptides expressed from nucleic acid constructs (i).

7. The method of claim 6, wherein said at least one therapeutic agent comprises a polypeptide obtained by eukaryotic or prokaryotic cellular recombinant DNA expression.

8. The method of claim 7, wherein said cellular recombinant DNA expression comprises recombinantly expressing polypeptide chains of subunits p35 and p40 of feline interleukin 12 from nucleic acid encoding same, to produce said polypeptide.

9. The method of claim 8, wherein subunits p35 and p40 are in equimolar concentration with respect to one another in said immunostimulant composition.

10. The method of claim 9, wherein said cellular recombinant DNA expression includes amplification of subunit p35 of feline IL-12 with a plasmid coding for human IL-12 p35.

11. The method of claim 10, wherein said nucleic acid comprises a nucleic acid construct from the group consisting of pMol-fIL12p35, pMol-fIL12p40, pCI-fIL-12, pCI-p40, and pCITE-p35.

12. The method of claim 11, wherein said immunostimulant composition comprises at least one antigen.

13. The method of claim 12, wherein said at least one antigen comprises gp140.

14. A method of making a therapeutic composition for treatment or prophylaxis of a disease or tumor associated with TH-1 deficiency, comprising recombinantly expressing, in eukaryotic or prokaryotic cells, polypeptide comprising polypeptide chains of subunits p35 and p40 of feline interleukin 12 from nucleic acid encoding same; extracting said polypeptide; and formulating said polypeptide in said therapeutic composition, wherein subunits p35 and p40 are in equimolar concentration with respect to one another.

15. The method of claim 14, wherein said nucleic acid is formed by steps including:
amplifying the 5′ region of cDNA of feline IL-12 p35 and the 3′ region of cDNA of human IL-12 p35, with primers yielding 3′ constructs overlapped with amplified 5′ constructs; separating strands of the constructs and subjecting same to PCR reaction, to yield said nucleic acid as a PCR reaction product.

16. The method of claim 15, wherein said nucleic acid is selected from the group consisting of nucleic acids having sequences with at least 95% homology to sequences of fIL12p40 (SEQ ID NO 1) and fIL12p35 (SEQ ID NO 2).

17. The method of claim 16, wherein said sequences with at least 95% homology to sequences of fIL12p40 (SEQ ID NO 1) and fIL12p35 (SEQ ID NO 2) are controlled by a promoter and terminator sequence that is active in Felidae.

18. The method of claim 17, further comprising incorporating at least one antigen in said therapeutic composition.

19. The method of claim 18, wherein said antigen comprises gp140.

20. The method of claim 19, wherein said nucleic acid comprises nucleic acid construct pCI-fIL-12.
